SEQUENCE LISTING (9 sequences)

SEQ ID NO: 1 - DNA, 427 bp, Homo sapiens
aggaaggtgcctctgtgtgtcccgtggccgctgtgacactgaccacacacctggggctggaaaataatactctctcccacagctctggagcgcaggagccatgggctgaggccagaatgtttgctccaggagcgtccctcgtggcccgttcaggtgcccagagttgcgggccttgcacgccttgtacgccttgttccctggcgcctcctccttccatgtgggtgtgcagcatcccgctccagggccttcagcctctgcgcccctcatctgctgatgcaggtgatggcatttaaggcccacctgggtactcctaggattcacctttatcaccgcatgagggagcattcccaggttccagggattagggataggactgggattcctttgggggctgctctcccgcccaccactgtgccggaatgtgatg

SEQ ID NO: 2 - DNA, 442 bp, Homo sapiens
aggaaggtgcctctgtgtgtcccgtggccgctgtgacactgaccacacacctggggctggaaaataatactctctcccacagctctggagcgcaggagccatgggctgaggccagaatgtttgctccaggagcgtccctcgtggcccgttcaggtgcccagagttgcgggccttgcacgccttgtacgccttgttccctggcgcctccttccctggcgcctcctccttccatgtgggtgtgcagcatcccgctccagggccttcagcctctgcgcccctcatctgctgatgcaggtgatggcatttaaggcccacctgggtactcctaggattcacctttatcaccgcatgagggagcattcccaggttccagggattagggataggactgggattcctttgggggctgctctcccgcccaccactgtgccggaatgtgatg

SEQ ID NO: 3 - DNA, 408 bp, Homo sapiens
aggaaggtgcctctgtgtgtcccgtggccgctgtgacactgaccacacacctggggctggaaaataatactctctcccacagctctggagcgcaggagccatgggctgaggccagaatgtttgctccaggagcgtccctcgtggcccgttcaggtgcccagagttgcgggccttgcacgccttgtacgccttgttccctggcgcctcctccttccatgtgggtgtgcagcatcccgctccagggccttcagcctctgcgcccctcatctgctgatgcaggtgatggcatttaagggattcacctttatcaccgcatgagggagcattcccaggttccagggattagggataggactgggattcctttgggggctgctctcccgcccaccactgtgccggaatgtgatg

SEQ ID NO: 4 - DNA, 418 bp, Homo sapiens
aggaaggtgcctctgtgtgtcccgtggccgctgtgacactgaccacacacctggggctggaaaataatactctctcccacagctctggagcgcaggagccatgggctgaggccagaatgtttgctccaggagcgtccctcgtggcccgttcaggtgcccagagttgcgggccttgcacgccttgttccctggcgcctcctccttccatgtgggtgtgcagcatcccgctccagggccttcagcctctgcgcccctcatctgctgatgcaggtgatggcatttaaggcccacctgggtactcctaggattcacctttatcaccgcatgagggagcattcccaggttccagggattagggataggactgggattcctttgggggctgctctcccgcccaccactgtgccggaatgtgatg

SEQ ID NO: 5 - DNA, 436 bp, Homo sapiens
aggaaggtgcctctgtgtgtcccgtggccgctgtgacactgaccacacacctggggctggaaaataatactctctcccacagctctggagcgcaggagccatgggctgaggccagaatgtttgctccaggagcgtccctcgtggcccgttcaggtgcccagagttgcgggccttgcacgccttgtacgccttgtacgccttgttccctggcgcctcctccttccatgtgggtgtgcagcatcccgctccagggccttcagcctctgcgcccctcatctgctgatgcaggtgatggcatttaaggcccacctgggtactcctaggattcacctttatcaccgcatgagggagcattcccaggttccagggattagggataggactgggattcctttgggggctgctctcccgcccaccactgtgccggaatgtgatg

SEQ ID NO: 6 - DNA, 22 bp, Artificial Sequence
  Synthetic
aggaaggtgcctctgtgtgtcc

SEQ ID NO: 7 - DNA, 20 bp, Artificial Sequence
  Synthetic
atcacattccggcacagtgg

SEQ ID NO: 8 - DNA, 20 bp, Artificial Sequence
  Synthetic
ggccagaatgtttgctccag

SEQ ID NO: 9 - DNA, 20 bp, Artificial Sequence
  Synthetic
acccaggtgggccttaaatg

(19) United States
(12) Patent Application Publication
(10) Pub. No.: US 2010/0184839 A1
(43) Pub. Date: Jul. 22, 2010

(54) ALLELIC POLYMORPHISM ASSOCIATED WITH DIABETES

(76) Inventors: Avishay Vaknin, Tel Aviv (IL); Pini Akiva, Moshav Chatzav (IL); Sarah Pollock, Tel Aviv (IL); Lily Bazak, Givatayim (IL); Erez Levanon, Brighton, MA (US); Tomer Zekharya, Petach Tikva (IL); Alex Golubev, Rehovot (IL); Gady Cojocaru, Ramat-Hasharon (IL); Liat Dassa, Tel Aviv (IL); Sarit Edelheit-Flohr, Tel Aviv (IL); Ronen Shemesh, Modi'in (IL); Anat Amir, Negev (IL); Yossi Cohen, Surrey (GB)

Correspondence Address: FENNEMORE CRAIG, 3003 NORTH CENTRAL AVENUE, SUITE 2600, PHOENIX, AZ, 85012, US

(21) Appl. No.: 12/598,286

(22) PCT Filed: Apr. 30, 2008
(86) PCT No.: PCT/IL08/00578

§ 371 Date: Mar. 1, 2010

Related U.S. Application Data
(60) Provisional application No. 60/914,927, filed on Apr. 30, 2007.

(51) Int. Cl. A61K 31/7088; C12Q 1/68; C07H 21/04

(52) U.S. Cl. 514/44 R; 435/6; 536/23.5; 536/24.33

(57) ABSTRACT

The invention relates to the identification of allelic polymorphism in a diabetes associated gene, particularly in a gene encoding phosphofructokinase (PFK) and use thereof for diagnosing diabetes predisposition and state and for predicting the response to a therapeutic agent.

### FIELD OF THE INVENTION

The present invention relates generally to the field of diagnosis and prognosis of diabetes mellitus, particularly, to the identification of allelic polymorphism in a diabetes-associated gene and use thereof for diagnosing diabetes, diabetes state and predisposition to diabetes as well as for predicting the response of a diabetic subject to a therapeutic agent.

### BACKGROUND OF THE INVENTION

Diabetes is a major cause of mortality and morbidity in the industrial world. Three forms of diabetes are known today: type 1 diabetes (T1D, or T1DM—type 1 diabetes mellitus), type 2 diabetes (T2D or T2DM), and gestational diabetes (occurring during pregnancy). All three types of diabetes have similar signs, symptoms, and consequences, but different causes and population distributions. In T1D, formerly known as insulin-dependent (IDDM), the pancreas fails to produce the insulin which is essential for survival, due to autoimmune destruction of the pancreatic beta cells. This form develops most frequently in children and adolescents, but is being increasingly diagnosed later in life. T2D, formerly named non-insulin-dependent (NIDDM), results from the body's inability to respond properly to the action of insulin produced by the pancreas, known as “insulin resistance” or “reduced insulin sensitivity”. T2D occurs most frequently in adults, but is being noted increasingly in adolescents as well. Gestational diabetes is similar to type 2 diabetes in that it involves insulin resistance; the hormones of pregnancy cause insulin resistance in those women predisposed to developing this condition. Additional form of diabetes is maturity onset diabetes of the young (MODY), which is similar to type 2 diabetes in its severity, leading to a form of insulin deficiency. However, whereas typical type 2 diabetic subjects are over-forty and over-weight, a MODY patient is typically in his teens or twenties and is thin.

Type 1 and type 2 diabetes are incurable chronic conditions, but have been treatable since insulin became medically available in 1921, and are nowadays usually managed with a combination of dietary treatment, tablets (in T2D) and, frequently, insulin supplementation. Gestational diabetes typically resolves with delivery.

Type 2 diabetes is a major public health problem of glucose homeostasis disorder affecting about 5% of the general population in the United States. The causes of the fasting hyperglycemia and/or glucose intolerance associated with this form of diabetes are not well understood.

Clinically, T2DM is a heterogeneous disorder characterized by chronic hyperglycemia. Subtypes of the T2DM can be identified based at least to some degree on the time of onset of the symptoms. The principal type of T2DM traditionally has onset in mid-life or later.

Diabetes can cause many complications, among them acute complications including hypoglycemia, ketoacidosis or nonketotic hyperosmolar state and coma that may occur if the disease is not adequately controlled. Serious long-term complications include atherosclerosis and cardiovascular disease (The risk of stroke is known to be markedly elevated in patients with T2DM), chronic renal failure (diabetic nephropathy is the main cause of dialysis adults in the developed world), retinal damage (which can lead to blindness and is the most significant cause of adult blindness in the non-elderly in the developed world), neuropathy or nerve damage (of several kinds), and microvascular damage, which may cause erectile dysfunction (impotence) and poor healing. Diabetic infections take longer to heal because of delayed macrophage introduction and diminished leukocyte migration, which causes a prolonged inflammatory phase in the wound healing cascade. Poor healing of wounds, particularly of the feet, can lead to gangrene which can require amputation—the leading cause of non-traumatic amputation in adults in the developed world. For these reasons, the disease may be associated with early morbidity and mortality.

Adequate treatment of diabetes, as well as increased emphasis on blood pressure control and lifestyle factors (such as smoking and keeping a healthy body weight), may improve the risk profile of most aforementioned complications.

T2DM may go unnoticed for years in a patient before diagnosis, as visible symptoms are typically mild or non-existent, without ketoacidotic episodes, and can be sporadic as well. However, the aforementioned severe complications can result from undiagnosed T2DM.

T2DM pathology is attributed to a combination of defective insulin secretion and insulin resistance or reduced insulin sensitivity. In the early stage the predominant abnormality is reduced insulin sensitivity, characterized by elevated levels of insulin in the blood. At this stage hyperglycemia can be reversed by a variety of measures and medications that improve insulin sensitivity or reduce glucose production by the liver, but as the disease progresses the impairment of insulin secretion worsens and therapeutic replacement of insulin often becomes necessary. There are numerous theories as to the exact cause and mechanism for this resistance, but central obesity (fat concentrated around the waist in relation to abdominal organs, and not subcutaneous fat, it seems) is known to predispose individuals for insulin resistance, possibly due to its secretion of adipokines that impair glucose tolerance. Abdominal fat is especially active hormonally.

Obesity is found in approximately 55% of patients diagnosed with type 2 diabetes. Other factors include aging (about 20% of elderly patients are diabetic in North America) and family history (Type 2 is much more common in those with close relatives who have had it), although in the last decade it has increasingly begun to affect children and adolescents, likely in connection with the greatly increased rates of childhood obesity.

The rapidly increasing prevalence of type 2 diabetes is thought to be due to environmental factors, such as increased availability of food and decreased opportunity and motivation for physical activity, acting on genetically susceptible individuals. The heritability of T2DM is one of the best established among common diseases and, consequently, genetic risk factors for T2DM have been the subject of intense research. Although the genetic causes of many monogenic forms of diabetes (maturity onset diabetes in the young, neonatal mitochondrial and other syndromic types of diabetes mellitus) have been elucidated, few variants leading to common T2DM have been clearly identified and individually confer only a small risk (odds ratio<1.1-1.25) of developing T2DM (Permutt, et al. 2005. J. Clin. Invest. 115, 1431-1439). Linkage studies have reported many T2DM-linked chromosomal regions and have identified putative, causative genetic variants in several gene including, e.g. CAPN10 (Horikawa et al. 2000. Nature Genet. 26, 163-175), ENPP1 (Meyre et al. 2005. Nature Genet. 37, 863-867), HNF4A (Love-Gregory et al. 2004 Diabetes 53, 1134-1140; Silander et al. 2004. Diabetes 53, 1141-1149) and ACDC (also called ADIPOQ, see Vasseur et al. 2002. Hum. Mol. Genet. 11, 2607-2614). In parallel, candidate-gene studies have reported many T2DM-associated loci, with coding variants in the nuclear receptor PPARG (P12A, see Altshuler et al. 2000. Nature Genet. 26, 76-80) and the potassium channel KCNJ11 (E23K, see Gloyn et al. 2003. Diabetes 52, 568-572) being among the very few that have been convincingly replicated. The strongest known T2DM association (odds ratio<1.7) was recently mapped to the transcription factor TCF7L2 and has been consistently replicated in multiple populations (Grant et al. 2006. Nature Genet. 38, 320-323).

T2DM is a complex disorder with wide range of metabolic defects that underlie the disease. The contribution of glucose metabolic pathways to the pathogenesis of the T2DM remains yet unclear.

The cellular fate of glucose begins with glucose transport and phosphorylation. Subsequent pathways of glucose utilization include aerobic and anaerobic glycolysis, glycogen formation, and conversion to other intermediates in the hexose phosphate or hexosamine biosynthesis pathways. Abnormalities in each pathway may occur in diabetic subjects; however, it is unclear whether perturbations in these pathways may lead to diabetes or are a consequence of the multiple metabolic abnormalities found in the disease.

Pancreatic β-cell glycolysis increases insulin secretion in a glucose concentration-dependent manner and could provide a link between impaired glucose metabolism and impaired insulin secretion (Henquin 2000. Diabetes 49, 1751-1760). Indeed, diminished glycolysis has been directly implicated in specific cases of type 2 diabetes. Deficiency in phosphofructokinase activity due to a heterozygous gene mutation has been reported in one Ashkenazi-Jewish type 2 diabetic family (Ristow et al. 1997. J Clin Invest 100, 2833-2841).

Phosphofructokinase-1 (PFK-1) is the most important regulatory enzyme of glycolysis. It is an allosteric enzyme made of 4 subunits and controlled by several activators and inhibitors. PFK-1 catalyzes the conversion of fructose 6-phosphate and ATP to fructose 1,6-bisphosphate and ADP. This step is subject to extensive regulation not only since it is irreversible, but also because after this step the original substrate is forced to proceed down the glycolytic pathway. This leads to a precise control of glucose, and the other monosaccharides galactose and fructose going down the glycolysis pathway. Before this enzyme's reaction, glucose-6-phosphate can potentially travel down the pentose phosphate pathway, or be converted to glucose-1-phosphate and polymerized into the storage form Glycogen.

In human, PFK exists in multimolecular forms, due to random tetramerization of three distinct subunits, M (muscle-type), L (liver-type), and P (platelet-type), each under a separate genetic control. Muscle cells consist of four identical subunits (M4), liver is consisting of L4, and red cell cab be found as M3L, M2L2, or ML3. A subunit composition with a higher proportion of platelet type subunits is found in platelets, brain and fibroblasts. Platelet PFK (PFKP) consists of P4 isozyme, whereas the predominant species of liver and muscle consists, respectively, of the L4 and M4 isozyme.

The expression pattern of PFKPs, as calculated by taking into account publicly available Affymetrix HU133 microarray data, suggests that this form is involved in glycolysis in many tissues.

Identifying genetic components underlying complex pathological syndromes like obesity and diabetes is an important goal of modern medicine. Genetic complexity also underlies stratification of patient populations presenting a single disease phenotype into sub-classes whose disorders might have differing genetic components or different responses to particular therapeutics.

In most cases, T2D results from a complex interaction of genetic, environmental, and demographic factors. Improved techniques of genetic analysis, especially candidate gene association studies and genome wide linkage analysis (genome wide scan, GWS), have enabled a search for genes that contribute to the development of T2D in the population.

No major single gene explaining the development of T2D has been identified. However, studies have demonstrated associations between various metabolic defects underlying the development of type 2 diabetes and polymorphisms in several susceptibility genes (e.g., peroxisome proliferator-activated receptors γ (PPARγ) and PPARγ-coactivator-1 (PGC-1)). Although more than a hundred candidate genes have been evaluated for T2D, only several have been widely replicated.

Gene-environment interactions have been found between PPARγ and birth weight affecting adult insulin sensitivity and between PPARγ and dietary fat intake influencing adult BMI. A gene-gene interaction has also been found between PPARγ and fatty acid binding protein 4, adipocyte (FABP4) affecting adult insulin sensitivity and body fat levels.

To date more than 30 GWSs have been reported to identify loci for T2D. Linked loci with at least suggestive LOD (Logarithm of odds) scores have been observed on every chromosome. Perhaps most striking is the lack of consistently linked loci. Demenais F et al (2003. Hum. Mol. Genet. 12, 1865-1873) applying the genome-search meta-analysis method (GSMA) to 4 published genome-wide scans of T2D from Caucasian populations (GIFT consortium, Finland, Sweden, UK and France) found evidence of susceptibility regions for T2D on chromosomes 1p13.1-q22, 2p22.1-p13.2, 6q21-q24.1, 12q21.1-q24.12, 16p12.3-q11.2 and 17p11.2-q22, which had modest or non-significant linkage in each individual study. This may serve to illustrate the heterogeneity of human T2D as well as the potential shortcomings of attempting to compare studies using different methodologies.

US Application Publication No. 2007/0059772 discloses genes, SNP markers and haplotypes of susceptibility or predisposition to T2D and sub-diagnosis of T2D. Methods and kits for diagnosis, prediction of clinical course and efficacy of treatments for T2D using polymorphisms in the T2D risk genes are also disclosed.

Heterozygosity in the human population is attributable to common variants of a given genetic sequence, and those skilled in the art have sought to comprehensively identify common genetic variations and to link such variations to medical conditions (see for example, Collins et al., Science 278:1580, 1997). Although these types of common genetic variations have identified causative mutations for monogenic disorders, they have not been as successful in identifying genetic components for complex, polygenic traits.

More recently, single nucleotide polymorphisms (SNPs) have been suggested as an alternative marker set. These single nucleotide substitutions or deletions are typically biallelic variants and occur at sufficient density to permit whole-genome association studies in outbred populations. However, SNP studies indicate that a sample size requirement of several thousand individuals would be required to obtain an adequate power for detecting disease-polymorphism association.

Haplotypes or diploid haplotype pairs constitute an alternative set of markers for an association test, and haplotype-based tests have been suggested for use in clinical studies. Nevertheless, haplotype-based tests require additional work relative to SNP-based tests, including direct sequencing or computational inference to identify haplotypes, and for now preclude less costly tests of pooled DNA.

Thus, there remains an unmeet need for markers based on medium and large scale variations in the human genome useful for diabetes diagnosis as well as for diagnostic therapy.

### SUMMARY OF THE INVENTION

The present invention provides means for diagnosing a subject for predisposition or susceptibility to diabetes and for the disease state as well as for theranostic studies, treatment selection and evaluation and treatment optimization. Particularly, the present invention provides diabetes-associated allelic polymorphisms and use thereof.

The present invention relates to polymorphism in the alleles of at least one gene encoding phosphofructokinase, particularly platelet phosphofructokinase (PFKP), associated with diabetes or predisposition to develop diabetes.

The “prediction” or “assessment” of risk to develop diabetes as used herein implies that the risk is either increased or reduced.

The present invention also relates to methods of estimating susceptibility or predisposition of an individual to diabetes, methods of determining the molecular subtype of diabetes as well as methods for prediction of clinical course and efficacy of treatments for diabetes using the polymorphisms in the at least one PFK gene.

As used herein, the term “diabetes” refers to diabetes of all types, including T1D, T2D, gestational diabetes and MODY.

The present invention is based in part on analyzing DNA repositories, comparing DNA samples of non-diabetic Caucasian Americans with no known diabetic relative (control population) with DNA samples from diabetes patients which are Caucasian Americans with at least one first degree known diabetic relative (disease population). Based on the data retrieved, the present invention now discloses the association of polymorphic alleles of a gene encoding phosphofructokinase (PFK), particularly PFKP, with diabetes or predisposition to diabetes and related disorders. The invention further discloses the design of diagnostic markers based on these polymorphism-disease associations and their use for diagnosing diabetes predisposition and/or susceptibility and diabetes prognosis. The markers of the invention may also be useful for theranostic studies including treatment selection, prediction of clinical course and efficacy of treatment, and for differentiating the responsiveness of individuals and/or populations to a specific drug.

According to one aspect, the present invention provides a method for diagnosing diabetes, disposition to diabetes or prognosis of diabetes or a condition related thereto in a subject comprising:
(a) providing a biological sample comprising genetic material from the subject;
(b) determining, in the genetic material, the presence of a nucleic acid sequence of at least one gene encoding phosphofructokinase (PFK); and
(c) analyzing the nucleic acid sequence for allelic polymorphism indicative of diabetes or predisposition to diabetes.

According to certain embodiments, PFK is an isoenzyme selected from the group consisting of muscle PFK (PFKM), liver PFK (PFKL) and platelet PFK (PFKP). According to certain currently preferred embodiments, the isoenzyme if PFKP.

According to certain embodiments, the allele indicative for diabetes or predisposition to diabetes is an allele having a higher appearance frequency in a diabetes population comparing to its appearance frequency in a non-diabetic population.

According to one embodiment, the allelic polymorphism indicative for diabetes or predisposition to diabetes comprises at least one allele having a nucleic acids sequence as set forth in any one of SEQ ID NO:1 and SEQ ID NO:2.

Single nucleotide polymorphisms, as well as insertions or deletions of one or two nucleotides can be found throughout the genome (see, for example, NCBI SNP database). Any such polymorphism within the various PFKP encoding alleles of the present invention, or within the polynucleotide sequences listed in Table 1 that retains the correlation between the PFKP allele and diabetes, should be encompassed within the present invention.

According to certain embodiments the allele is GVAR237_ref having a nucleic acids sequence as set forth in SEQ ID NO:1 or a homolog thereof, said GVAR237 allele has a correlation with reduced risk to diabetes. According to other embodiments, the GVAE237-ref allele further comprises at least one SNP and/or at least one insertion or deletion of one or two nucleotides, said allele retaining the correlation with reduced risk for diabetes. According to yet other embodiments the allele is GVAR237_ins having a nucleic acid sequence as set forth in SEQ ID NO:2 or a homolog thereof, said allele has a correlation with increased risk to diabetes. According to further embodiments, the GVAR237_ins comprises at least one SNP and/or at least one insertion or deletion of one or two nucleotides, said allele retaining the correlation of GVAR237_ins allele with increased risk for diabetes, susceptibility or predisposition to diabetes.

According to certain embodiments, the methods of the invention are for diagnosing a subtype of the disease. According to one embodiment, diabetes subtype is selected from the group consisting of type 1 diabetes, type 2 diabetes, gestational diabetes and maturity onset diabetes of the young (MODY).

The methods of the present invention allow the accurate diagnosis of diabetes at or before disease onset, thus reducing or minimizing the debilitating effects of diabetes. The method can be applied in persons who are free of clinical symptoms and signs of the disease, in those who have family history of having the disease or in those who have elevated level or levels of additional risk factors to develop diabetes.

Diagnostic tests, identifying the risk of diabetes by defining genetic factors contributing to diabetes, can be used together with or independent of information regarding known clinical risk factors to define an individual's risk relative to the general population. Better means for identifying those individuals at risk for diabetes should lead to better preventive and treatment regimens, including more aggressive management of the risk factors for diabetes, particularly diabetes type 2, including cigarette smoking, hypercholesterolemia, elevated LDL cholesterol, low HDL cholesterol, elevated blood pressure (BP), obesity, lack of physical activity, and inflammatory components as reflected by increased C-reactive protein levels or other inflammatory markers.

Such additional information can be obtained from blood measurements, clinical examination and questionnaires. The blood measurements include but are not restricted to the determination of plasma or serum cholesterol and high-density lipoprotein cholesterol. The information to be collected by questionnaire includes information concerning gender, age, family and medical history such as the family history of obesity and diabetes. Clinical information collected by examination includes e.g. information concerning height, weight, hip and waist circumference and other measures of adiposity and obesity. Information on genetic risk may be used by physicians to help convince particular patients to adjust life style (e.g. to stop smoking, to reduce caloric intake, to increase exercise). Finally, preventive measures aimed at lowering blood pressure such as reduction of weight, intake of salt and alcohol can be both better motivated to the patients who are at an elevated risk of diabetes and selected on the basis of the molecular diagnosis of diabetes according to the teaching of the present invention.

Thus, according to certain embodiments, the methods of the present invention are for identifying subjects having altered risk for developing diabetes and related disorders.

According to other embodiments, the diabetes-related disorder or condition is selected from the group consisting of obesity including morbid obesity; Prader-Willi syndrome; Hyperphagia and impaired satiety; anorexia; metabolic disorders; Endocrine disorders; gastrointestinal diseases; eating disorders; Wolfram syndrome; Alstrom syndrome; mitochondrial myopathy with diabetes; MED-IDDM syndrome; Ipex-linked syndrome; Congenital generalized lipodystrophy (type 2) (Cgl2), or Berardinelli-Seip syndrome; and Schmidt syndrome.

According to further embodiments, the methods are for selecting efficient and safe therapy or for monitoring the effect of a therapy on the disease.

According to yet further embodiments, the methods of the present invention are also useful for assessing drug effectiveness, including drug action, drug responsiveness by the subject and drug side effects.

The invention further provides a method of diagnosing susceptibility to diabetes in a population. This method comprises screening for diabetes-associated PFK variant alleles that are more frequently present in a population susceptible to said disease, compared to the frequency of its presence in the general population, wherein the presence of at least one diabetes-associated PFK variant allele is indicative of a susceptibility to diabetes. The “disease-associated variant allele” may also be associated with a reduced rather than increased risk of having diabetes. A “disease-associated alleles” is intended to include one or a combination of the allelic polymorphism described herein that show high correlation to diabetes.

Those skilled in the art will readily recognize that determining the presence of at least one polymorphic allele in a sample containing an individual's genetic material can be done by any method or technique as is known to a person skilled in the art, including, but not limited to, PCR, restriction fragment length polymorphism (RFLP), hybridization, direct sequencing and any combination thereof. As is obvious in the art, the presence of a specific allele can be determined from either nucleic acid strand or from both strands.

Thus, according to additional aspect, the present invention provides a primer pair comprising a pair of isolated oligonucleotides capable of amplifying any one of the polymorphic alleles of the PFK gene having a nucleic acid sequence as set forth in SEQ ID NO:1-2.

According to one embodiment, the pair of isolated oligonucleotides comprises a forward primer having the nucleic acids sequence AGGAAGGTGCCTCTGTGTGTCC (SEQ ID NO:6) and a reverse primer having the nucleic acid sequence ATCACATTCCGGCACAGTGG (SEQ ID NO:7). According to another embodiment the pair of isolated oligonucleotides comprises a forward primer having the nucleic acids sequence GGCCAGAATGTTTGCTCCAG (SEQ ID NO:8) and a reverse primer having the nucleic acid sequence ACCCAGGTGGGCCTTAAATG (SEQ ID NO:9).

As described hereinabove, one of the objectives of the present invention is the prediction of those at higher risk of developing diabetes. Better means for identifying those individuals at risk for the disease should lead to better preventive and treatment regimens, including more aggressive management of the risk factors for diabetes as well as preventing long-term complications associated with diabetes.

Long-term complications associated with diabetes include atherosclerosis; cardiovascular diseases, including peripheral vascular disease, congestive heart failure, coronary artery disease, myocardial infarction, and sudden death; chronic renal failure due to diabetic nephropathy; retinal damage due to diabetic retinopathy; nonproliferative diabetic retinopathy; proliferative diabetic retinopathy neuropathy, including polyneuropathy, mononeuropathy, and/or autonomic neuropathy; gastrointestinal dysfunction, including delayed gastric emptying (gastroparesis) and altered small- and large-bowel motility (constipation or diarrhea); genitourinary dysfunction, including erectile dysfunction, cystopathy and female sexual dysfunction; dermatological manifestations including poor healing of wounds and diabetic dermopathy; and lower extremity complications like foot ulcer and gangrenes.

A further object of the invention is to provide a method for molecular diagnosis of a disease. The genetic etiology of a disease in an individual will provide information of the molecular etiology of this disease. When the molecular etiology is known, the therapy can be selected on the basis of this etiology. For example, the drug that is likely to be effective can be more directly selected without the need of intensive trial and error clinical trials. The teaching of the present invention also enables the selection of human subjects for studies testing the effects of a drug on the disease, including testing the effects of known, in use drugs as well as examining the effect of a new drug during the clinical trials of its development.

Thus, according to a further aspect the present invention provides a method for monitoring the effect of a therapeutic agent useful in the prevention or treatment of a diabetes comprising:
(a) providing a therapeutically effective amount of the agent to a subject having within its genome at least one diabetes-associated polymorphic allele having a nucleic acid sequence set forth in any one of SEQ ID NOs:1-2;
(b) determining the effect of said therapeutic agent on at least one phenotypic characteristic of diabetes;

wherein an agent altering the at least one phenotypic characteristic is considered useful in preventing or treating diabetes.

As used herein, the term “phenotypic characteristic of diabetes” includes, but is not limited to, hyperglycemia and disorders associated thereto, including acute complications such as ketoacidosis and nonketotic hyperosmolar coma and long term complications. Long term complications associated with diabetes include atherosclerosis; cardiovascular diseases, including peripheral vascular disease, congestive heart failure, coronary artery disease, myocardial infarction, and sudden death; chronic renal failure due to diabetic nephropathy; retinal damage due to diabetic retinopathy; nonproliferative diabetic retinopathy; proliferative diabetic retinopathy neuropathy, including polyneuropathy, mononeuropathy, and/or autonomic neuropathy; gastrointestinal dysfunction, including delayed gastric emptying (gastroparesis) and altered small- and large-bowel motility (constipation or diarrhea); genitourinary dysfunction, including erectile dysfunction, cystopathy and female sexual dysfunction; dermatological manifestations including poor healing of wounds and diabetic dermopathy; and lower extremity complications like foot ulcer and gangrenes.

According to certain embodiments, the agent is provided to a plurality of subjects, each comprising within it genome a different variant allele. In these embodiments, the method further comprises analyzing the effect of the therapeutic agent with regard to allelic combination of the plurality of subjects as to predict the linkage between specific allelic combination and effect of said therapeutic agent, particularly a drug.

According to yet further aspect the present invention provides a method for monitoring the responsiveness of a subject to a candidate therapeutic agent for treating diabetes comprising:
(a) providing a biological sample comprising genetic material from the subject;
(b) determining, in the genetic material, the presence of at least one polymorphic allele present within a gene encoding PFK, wherein the allele has a nucleic acid sequence as set forth in any one of SEQ ID NO:1 and SEQ ID NO:2;
(c) administering to said subject a therapeutically effective amount of the candidate agent; and
(d) determining the effect of said candidate agent on said subject;

wherein having a detectable effect indicates said subject as responsive to said candidate agent.

According to certain embodiments, determining the effect of the candidate agent comprises determining its effect on at least one characteristic phenotype of diabetes.

According to other embodiments, determining the effect of the candidate agent comprises comparing the level of expression or activity of a protein, mRNA or genomic DNA or a biological reaction or pathway related thereto in the sample provided from the subject before administering the candidate agent and in a sample obtained from said subject after administration of said candidate agent, wherein an agent altering said level of expression or activity is considered useful in preventing or treating diabetes.

According to certain embodiments, the agent is administered to a plurality of subjects and the method further comprises analyzing the effect of the therapeutic agent with regard to allelic combination of the plurality of subjects as to predict the linkage between specific allelic combination and the effect of the therapeutic agent.

According to still other embodiments, determining the effect comprises determining the degree of responsiveness of the subject. According to further embodiments, the effect of the candidate agent is a side-effect, including adverse effect, and determining the effect includes determining the degree of said side effect.

Kits useful for use according to the methods of the present invention are also provided.

According to yet additional aspect, the present invention provides a kit for risk assessment, diagnosis or prognosis of diabetes or a related condition comprising reagent, materials and protocols capable of identifying the diabetes-associated polymorphic alleles described herein.

According to one embodiment, the kit comprises reagents and material capable of detecting at least one of the polymorphic alleles of the PFKP gene of the present invention.

According to certain embodiments, the material comprises a primer pair capable of amplifying a polymorphic alleles of the PFK gene having a nucleic acid sequence as set forth in any one of SEQ ID NOs:1-2. According to certain currently preferred embodiments, the pair of isolated oligonucleotides comprises a forward primer having the nucleic acids sequence AGGAAGGTGCCTCTGTGTGTCC (SEQ ID NO:6) and a reverse primer having the nucleic acid sequence ATCACATTCCGGCACAGTGG (SEQ ID NO:7). According to another embodiment the pair of isolated oligonucleotides comprises a forward primer having the nucleic acids sequence GGCCAGAATGTTTGCTCCAG (SEQ ID NO:8) and a reverse primer having the nucleic acid sequence ACCCAGGTGGGCCTTAAATG (SEQ ID NO:9).

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 demonstrates the sequence of GVAR237_ins allele, showing the insertion of 15 nucleotides in bold Italian font.

FIG. 2 shows an example of a mixed sequence (marked as “mix”) and the separated reference and insertion alleles (marked as “ref” and “ins”, accordingly). The sequences are the reverse complement of the alleles in Table 1 and only the variable region is shown, with the insertion shown in bold and Italian font.

FIG. 3 shows gel electrophoresis demonstrating results for 20 samples from patients homozygous or heterozygous to GVAR237_ref and GVAR237_ins alleles.

### DESCRIPTION OF PREFERRED EMBODIMENTS

The invention relates to the identification of genetic variation related to diabetes, particularly to the identification of allelic polymorphism in a disease-associated gene. The present invention provides methods for diagnosing disease predisposition and disease state as well as methods for predicting the response to a therapeutic agent. The invention further provides kits useful for practicing of the present invention.

### Methodology Taken to Uncover the Sequences of the Present Invention

The GeneVa™ platform was used to search for genomic variations linked to diabetes. The platform contains three main components: (a) A large database of insertions and deletions in the human genome; (b) A system linking structural genomic variations, genes, diseases and drugs; and (c) A sequencing-based genotyping method for insertions and deletions.

The database platform consists of over 200,000 fine scale variations of size in the range of 15-500 bp. More than 40% of the fine scale variations, are located within known genes and thousands are located in drug target genes and in drug target interacting genes. The database was created by analyzing all human genome sequencing fragments (over 230 million sequences) and public and proprietary EST (expressed sequence tags) databases. Human genome fragments were downloaded from NCBI Trace archive and public ESTs were downloaded from NCBI GenBank.

A bioinformatics analysis system was developed to link genes with diseases, integrating disparate data sources such as published papers, gene expression microarray experiments, pathway databases, and pharma related databases. The system was used to find genes related to diabetes, and the predicted variations within these genes. The variations are then filtered according to their potential effect on the gene product, such as whether they are in a coding exon, a regulatory region, a conserved region etc. The key to genotyping insertions and deletions by sequencing is having an accurate method to analyze chromatograms in the heterozygote state. A mixed chromatogram decomposition algorithm was used to handle complicated cases, including alleles appearing only in the heterozygote state, complex chromatograms representing unpredicted allele combinations and multi-allelic sites. This method was used to genotype fine scale variations.

### Genetic Variation

The present invention discloses the association of diabetes and related disorders with allelic polymorphism within phosphofructokinase (PFK) genes, particularly within the PFKP gene, as described in details herein below.

The term “gene” as used herein refers to an entirety containing all regulatory elements located both upstream and downstream as well as within of a polypeptide-encoding sequence of a gene and entire transcribed region of a gene including 5′ and 3′ untranslated regions of mRNA and the entire polypeptide encoding sequence including all exon and intron sequences (also alternatively spliced exons and introns) of a gene.

As used herein, the term “Polymorphism” refers to the coexistence in a population of more than one form of a gene or portion (e.g., allelic variant) thereof. A portion of a gene of which there are at least two different forms, i.e., two different nucleotide sequences, is referred to as a polymorphic site. A specific genetic sequence at a polymorphic site is an allele. A polymorphic site can be a single nucleotide, the identity of which differs in different alleles. A polymorphic site can also be several nucleotides long. A polymorphism is thus then said to be “allelic,” in that, due to the existence of the polymorphism, some members of a population carry a gene with one sequence, whereas other members carry a second, slightly different sequence. In the simplest case, only one variant of the sequence may exist, and the polymorphism is said to be diallelic. The occurrence of alternative mutations can give rise to triallelic polymorphisms, etc. An allele may be referred to by the nucleotide(s) that comprise the sequence difference.

### Disease-Associated Polymorphic Alleles

According to certain embodiments, the present invention discloses polymorphic alleles within the gene encoding platelet phosphofructokinase (PFKP), described in Table 1 hereinbelow:

TABLE 1

| | |
|---|---|
| Variation name | Allele |

| | |
|---|---|
| GVAR237_ref | AGGAAGGTGCCTCTGTGTGTCCCGTGGCCGCTGTGACACTGACCACACA |
| (SEQ ID NO: 1) | CCTGGGGCTGGAAAATAATACTCTCTCCCACAGCTCTGGAGCGCAGGAG |
| | CCATGGGCTGAGGCCAGAATGTTTGCTCCAGGAGCGTCCCTCGTGGCCC |
| | GTTCAGGTGCCCAGAGTTGCGGGCCTTGCACGCCTTGTACGCCTTGTTC |
| | CCTGGCGCCTCCTCCTTCCATGTGGGTGTGCAGCATCCCGCTCCAGGGC |
| | CTTCAGCCTCTGCGCCCCTCATCTGCTGATGCAGGTGATGGCATTTAAG |
| | GCCCACCTGGGTACTCCTAGGATTCACCTTTATCACCGCATGAGGGAGC |
| | ATTCCCAGGTTCCAGGGATTAGGGATAGGACTGGGATTCCTTTGGGGGC |
| | TGCTCTCCCGCCCACCACTGTG CCGGAATGTGATG |
| GVAR237_ins | AGGAAGGTGCCTCTGTGTGTCCCGTGGCCGCTGTGACACTGACCAC |
| (SEQ ID NO: 2) | ACACCTGGGGCTGGAAAATAATACTCTCTCCCACAGCTCTGGAGCG |
| | CAGGAGCCATGGGCTGAGGCCAGAATGTTTGCTCCAGGAGCGTCCC |
| | TCGTGGCCCGTTCAGGTGCCCAGAGTTGCGGGCCTTGCACGCCTTG |
| | TACGCCTTGTTCCCTGGCGCCTCCTTCCCTGGCGCCTCCTCCTTCC |
| | ATGTGGGTGTGCAGCATCCCGCTCCAGGGCCTTCAGCCTCTGCGCC |
| | CCTCATCTGCTGATGCAGGTGATGGCATTTAAGGCCCACCTGGGTA |
| | CTCCTAGGATTCACCTTTATCACCGCATGAGGGAGCATTCCCAGGT |
| | TCCAGGGATTAGGGATAGGACTGGGATTCCTTTGGGGGCTGCTCTC |
| | CCGCCCACCACTGTGCCGGAATGTGATG |
| GVAR237_A2 | AGGAAGGTGCCTCTGTGTGTCCCGTGGCCGCTGTGACACTGACCAC |
| (SEQ ID NO: 3) | ACACCTGGGGCTGGAAAATAATACTCTCTCCCACAGCTCTGGAGCG |
| | CAGGAGCCATGGGCTGAGGCCAGAATGTTTGCTCCAGGAGCGTCCC |
| | TCGTGGCCCGTTCAGGTGCCCAGAGTTGCGGGCCTTGCACGCCTTG |
| | TACGCCTTGTTCCCTGGCGCCTCCTCCTTCCATGTGGGTGTGCAGC |
| | ATCCCGCTCCAGGGCCTTCAGCCTCTGCGCCCCTCATCTGCTGATG |
| | CAGGTGATGGCATTTAAGGGATTCACCTTTATCACCGCATGAGGGA |
| | GCATTCCCAGGTTCCAGGGATTAGGGATAGGACTGGGATTCCTTTG |
| | GGGGCTGCTCTCCCGCCCACCACTGTGCCGGAATGTGATG |
| GVAR237_A3 | AGGAAGGTGCCTCTGTGTGTCCCGTGGCCGCTGTGACACTGACCAC |
| (SEQ ID NO: 4) | ACACCTGGGGCTGGAAAATAATACTCTCTCCCACAGCTCTGGAGCG |
| | CAGGAGCCATGGGCTGAGGCCAGAATGTTTGCTCCAGGAGCGTCCC |
| | TCGTGGCCCGTTCAGGTGCCCAGAGTTGCGGGCCTTGCACGCCTTG |
| | TTCCCTGGCGCCTCCTCCTTCCATGTGGGTGTGCAGCATCCCGCTC |
| | CAGGGCCTTCAGCCTCTGCGCCCCTCATCTGCTGATGCAGGTGATG |
| | GCATTTAAGGCCCACCTGGGTACTCCTAGGATTCACCTTTATCACC |
| | GCATGAGGGAGCATTCCCAGGTTCCAGGGATTAGGGATAGGACTGG |
| | GATTCCTTTGGGGGCTGCTCTCCCGCCCACCACTGTGCCGGAATGT |
| | GATG |
| GVAR237_A4 | AGGAAGGTGCCTCTGTGTGTCCCGTGGCCGCTGTGACACTGACCACA |
| (SEQ ID NO: 5) | CACCTGGGGCTGGAAAATAATACTCTCTCCCACAGCTCTGGAGCGCA |
| | GGAGCCATGGGCTGAGGCCAGAATGTTTGCTCCAGGAGCGTCCCTCG |
| | TGGCCCGTTCAGGTGCCCAGAGTTGCGGGCCTTGCACGCCTTGTACG |
| | CCTTGTACGCCTTGTTCCCTGGCGCCTCCTCCTTCCATGTGGGTGTG |
| | CAGCATCCCGCTCCAGGGCCTTCAGCCTCTGCGCCCCTCATCTGCTG |
| | ATGCAGGTGATGGCATTTAAGGCCCACCTGGGTACTCCTAGGATTCA |
| | CCTTTATCACCGCATGAGGGAGCATTCCCAGGTTCCAGGGATTAGGG |
| | ATAGGACTGGGATTCCTTTGGGGGCTGCTCTCCCGCCCACCACTGTG |
| | CCGGAATGTGATG |

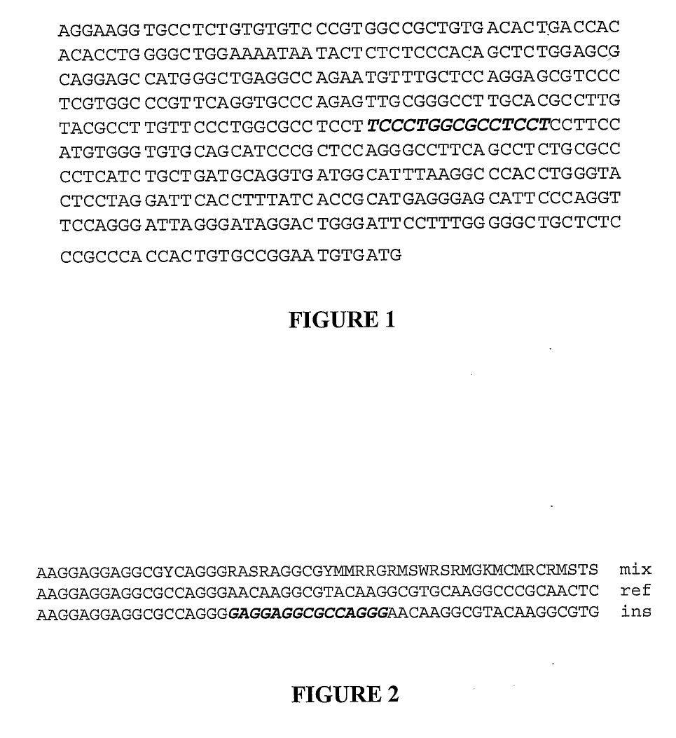

The sequence corresponding to the sequence of allele GVAR237_ref appears in NCBI reference genome within an intron of the PFKP gene (NCBI Accession number: ref|NT_{—}077567.3|Hs10_{—}77616, from 3110627 to 3111053). The sequence of the present invention corresponding to the sequence of allele GVAR237_ins contains insertion of 15 nucleotide relative to the GVAR237_ref, marked as bold and Italian font in FIG. 1.

### Use of the Polymorphic Alleles

According to certain embodiments, the polymorphic alleles of the invention are useful as markers for the risk assessment, diagnosis and prognosis of a certain disease. According to other embodiments, the markers are useful for theranostic studies, for selecting treatment and monitoring the responsiveness of a subject to the treatment and the efficacy of the treatment for prevention and treatment of diabetes, for prediction of clinical course and efficacy of a treatment, and as surrogate markers.

As used herein the terms “predisposition” and “susceptibility” are used herein interchangeably, referring to an increased probability of a subject to develop the phenotypic characteristics of diabetes, including all diabetes subtypes.

The markers are alleles of a gene associated with diabetes. According to the teaching of the present invention, the gene is PFK, particularly PFKP. As used herein, the term “disease associated gene” “diabetes-associated gene” “a gene associated with diabetes”, or “at risk diabetes gene” are used interchangeably, and refer to the association between a gene encoding a protein found to be associated with at least one disease or disorder, using publicly available as well as proprietary databases.

A nucleotide position in genome at which more than one sequence is possible in a population, is referred to herein as a “polymorphic site”. Where a polymorphic site is a single nucleotide in length, the site is referred to as a single nucleotide polymorphism (SNP). For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a particular gene has a reference nucleotide sequence e.g. from the NCBI reference genome (www.ncbi.nlm.nih.gov). Alleles that differ from the reference are referred to as “variant alleles”. The polypeptide encoded by the reference nucleotide sequence is the “reference” polypeptide with a particular reference amino acid sequence, and polypeptides encoded by variant alleles are referred to as “variant” polypeptides with variant amino acid sequences.

The differences between the reference and variant allele are not necessary reflected in the reference and variant polypeptides. Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal mRNA splicing; the deletion of several nucleotides, result in a deletion of one or more amino acids encoded by the nucleotides; the insertion of several nucleotides, such as by unequal recombination or gene conversion, result in an interruption of the coding sequence of a reading frame; duplication of all or a part of a sequence; transposition; or a rearrangement of a nucleotide sequence, as described in detail above. Such sequence changes alter the polypeptide encoded by the gene found to be associated with the disease. For example, a nucleotide change resulting in a change in polypeptide sequence can dramatically alter the physiological properties of the polypeptide resulting in altered activity, distribution and stability or otherwise affect on properties of the polypeptide.

Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of the disease-associated gene.

A “haplotype” as described herein, refers to any combination of genetic markers (“alleles”). A haplotype can comprise two or more alleles and the length of a genome region comprising a haplotype may vary from few hundred bases up to hundreds of kilobases. As it is recognized by those skilled in the art the same haplotype can be described differently by determining the haplotype defining alleles from different nucleic acid strands. In the context of the present invention a haplotype preferably refers to a combination of alleles found in a given individual and which may be associated with a phenotype.

It is to be understood that the diabetes associated alleles described in the present invention may be associated with other “polymorphic sites” located in additional genes associated with diabetes. These other disease associated polymorphic sites may be either equally useful as genetic markers or even more useful as causative variations explaining the observed association of at-risk alleles of this invention to diabetes.

According to certain embodiments of the invention, an individual who is at risk for diabetes is an individual in whom an allele of a diabetes associated gene is identified. According to certain embodiments, the significance of the risk is measured by a percentage. In one embodiment, a significant increase or reduction in risk is at least about 20%, including but not limited to about 25%, 30%, 35%,40%, 45%, 50%, 55%, 60%, 65%, 70%, 75%, 80%, 85%, 90%, 95% and 98%. In another embodiment, a significant increase in risk is at least about 50%. It is understood however, that identifying whether a risk is medically significant may also depend on a variety of factors, including the specific diabetes type, the allele or the haplotype, and often, environmental factors.

### NAT Assays

One aspect of the invention includes the identification and detection of diabetes—associated allelic polymorphism.

Detection of a nucleic acid of interest in a biological sample may optionally be effected by NAT-based assays, which involve nucleic acid amplification technology, such as PCR for example (or variations thereof such as real-time PCR for example).

As used herein, the term “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods (See generally Kwoh et al., 1990, Am. Biotechnol. Lab. 8:14). Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, Current Protocols in Molecular Biology Volumes I-III).

The terminology “primer pair” or “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (i.e. DNA or RNA) for practicing the present invention may be obtained according to methods well known in the art.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 nucleotides, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than 7° C., alternatively less than 5° C., or less than 4° C., typically less than 3° C., more typically between 3° C. and 0 ° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified”.

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation and ligation amplify a short segment of DNA. LCR has also been used in combination with PCR to achieve enhanced detection of single-base changes: see for example Segev, PCT Publication No. W09001069 A1 (1990).

Self-Sustained Synthetic Reaction (3SR/NASBA): The self-sustained sequence replication reaction (3 SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo-and deoxyribonucleoside triphosphates, the target sequence undergoes repeated rounds of transcription, cDNA synthesis and second-strand synthesis to amplify the area of interest. The use of 3 SR to detect mutations is kinetically limited to screening small segments of DNA (e.g., 200-300 base pairs).

Q-Beta (Qβ) Replicase: In this method, a probe which recognizes the sequence of interest is attached to the RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37° C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each of the methods cannot be used at high temperature (i.e., >55° C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

Additional NAT tests are Fluorescence In Situ Hybridization (FISH) and Comparative Genomic Hybridization (CGH). Fluorescence In Situ Hybridization (FISH)—The test uses fluorescent single-stranded DNA probes which are complementary to the DNA sequences that are under examination (genes or chromosomes). These probes hybridize with the complementary DNA and allow the identification of the chromosomal location of genomic sequences of DNA.

Comparative Genomic Hybridization (CGH)—allows a comprehensive analysis of multiple DNA gains and losses in entire genomes. Genomic DNA from the tissue to be investigated and a reference DNA are differentially labeled and simultaneously hybridized in situ to normal metaphase chromosomes. Variations in signal intensities are indicative of differences in the genomic content of the tissue under investigation.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. A similar 3′-mismatch strategy is used with greater effect to prevent ligation in the LCR, wherein any mismatch effectively blocks the action of the thermostable ligase.

The direct detection method according to various embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding may also be increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of small differences between like sequences, the requirements of the analysis are often at the highest level of resolution. For cases in which the position of the variation in question is known in advance, several methods have been developed for examining small changes without direct sequencing. For example, if a mutation of interest happens to fall near or within a restriction recognition sequence, a change in the pattern of digestion can be used as a diagnostic tool (e.g., restriction fragment length polymorphism [RFLP] analysis).

RFLP analysis suffers from low sensitivity and requires a large amount of sample. When RFLP analysis is used for the detection of small mutations, it is, by its nature, limited to the detection of only those changes which fall near or within a restriction sequence of a known restriction endonuclease. Moreover, the majority of the available enzymes has 4 to 6 base-pair recognition sequences, and cleaves too frequently for many large-scale DNA manipulations. Thus, RFLP is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity.

Allele specific oligonucleotide (ASO): If the change is not in a recognition sequence, then allele-specific oligonucleotides (ASOs) can be designed to hybridize in proximity to the mutated sequence, such that a primer extension or ligation event can be used as the indicator of a match or a miss-match. Hybridization with radioactively labeled allelic specific oligonucleotides (ASO) also has been applied to the detection of specific mutations. The method is based on the differences in the melting temperature of short DNA fragments, even when differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretic ally resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

According to a presently preferred embodiment of the present invention the step of searching for any of the nucleic acid sequences described here in a DNA sample is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for diabetes and/or pathological condition both rapidly and easily.

### Hybridization Assays

Detection of a nucleic acid of interest in a biological sample may optionally be effected by hybridization-based assays using an oligonucleotide probe. As used herein, “probe” is oligonucleotide that hybridizes in a base-specific manner to a complementary strand of nucleic acid molecules. By “base specific manner” is meant that the two sequences must have a degree of nucleotide complementarity sufficient for the primer or probe to hybridize. Accordingly, the probe sequence is not required to be perfectly complementary to the sequence of the template. Non-complementary bases or modified bases can be interspersed into the probe, provided that base substitutions do not inhibit hybridization. The nucleic acid template may also include “nonspecific sequences” to which the probe has varying degrees of complementarity.

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of an allele of interest (i.e.,

DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described allelic nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

More generally, hybridization of short nucleic acids (below 200 bp in length, e.g. 17-40 by in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S. Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif.] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNase A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As is commonly known, radioactive nucleotides can be incorporated into probes of the invention by several methods. Non-limiting examples of radioactive labels include ³H, ¹⁴C, ³²P, and ³⁵S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

### Diagnostic Assays

The markers, probes and primers described herein can be used in methods and kits for risk assessment, diagnosis or prognosis of diabetes or condition associated with diabetes in a subject.

According to one embodiment, diagnosis of risk or susceptibility to diabetes or a condition associated thereto is made by detecting one or several of polymorphic alleles described in the present invention in the subject's nucleic acid. Diagnostically, the most useful polymorphic alleles are those which are associated with altering the polypeptide encoded by the diabetes-associated gene due to a frame shift; due to a premature stop codon; due to an amino acid change; or due to abnormal mRNA splicing. Nucleotide changes resulting in a change in polypeptide sequence in many cases alter the physiological properties of a polypeptide by resulting in altered activity, distribution and stability or otherwise affect on properties of a polypeptide. Other diagnostically useful polymorphic alleles are those affecting transcription of the diabetes associated gene or translation of it's mRNA due to altered tissue specificity, due to altered transcription rate, due to altered response to physiological status, due to altered translation efficiency of the mRNA and due to altered stability of the mRNA.

In diagnostic assays determination of the nucleotides present in one or several of the diabetes associated polymorphic alleles of this invention in an individual's nucleic acid can be done by any method or technique which can accurately determine nucleotides present in a polymorphic site as is known to a person skilled in the art and as described hereinabove.

According to other embodiments of the invention, diagnosis of a susceptibility to diabetes can be assessed by examining transcription of one or several diabetes associated alleles. Alterations in transcription can be assessed by a variety of methods described in the art, including e.g. hybridization methods, enzymatic cleavage assays, RT-PCR assays and microarrays. A test sample from an individual is collected and the alterations in the transcription of the diabetes associated alleles are assessed from the RNA present in the sample. Altered transcription is diagnostic for a susceptibility to diabetes.

According to further embodiments of the invention, diagnosis of a susceptibility to diabetes can also be made by examining expression and/or structure and/or function of a polypeptide encoded by the alleles of the invention. A test sample from an individual is assessed for the presence of an alteration in the expression and/or an alteration in structure and/or function of the polypeptide encoded by the diabetes risk gene, or for the presence of a particular polypeptide variant (e.g., an isoform) encoded by the diabetes risk gene. An alteration in expression of a polypeptide encoded by the diabetes risk gene can be, for example, quantitative (an alteration in the quantity of the expressed polypeptide, i.e., the amount of polypeptide produced) or qualitative (an alteration in the structure and/or function of the polypeptide i.e. expression of a mutant polypeptide or of a different splicing variant or isoform).

Alterations in expression and/or structure and/or function of a diabetes susceptibility polypeptide can be determined by various methods known in the art, e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition in a control sample and an alteration can be assessed either directly from the diabetes susceptibility polypeptide or its fragment or from substrates and reaction products of said polypeptide. A control sample is a sample that corresponds to the test sample (e.g., is from the same type of cells), and is from an individual who is not affected by the disease. An alteration in the expression or composition of a polypeptide encoded by a diabetes susceptibility gene of the invention in the test sample, as compared with the control sample, is indicative of a susceptibility to diabetes.

Western blotting analysis, using an antibody that specifically binds to a polypeptide encoded by an allele of the present invention or an antibody that specifically binds to a polypeptide encoded by a reference gene can be used to identify the presence or absence in a test sample of a particular polypeptide encoded by a polymorphic allele of the invention. The presence of a polypeptide encoded by a polymorphic allele, or the absence of a polypeptide encoded by a reference gene, is diagnostic for a susceptibility to diabetes.

The invention also pertains to methods of diagnosing risk or a susceptibility to diabetes in a population, comprising screening for a diabetes-associate allele that is more frequently present in a diabetes-affected population compared to the frequency of its presence in a healthy population (control), wherein the presence of the allele is indicative of risk or susceptibility to diabetes.

Yet in another embodiment, a susceptibility to diabetes can be diagnosed by assessing the status and/or function of biological networks and/or metabolic pathways related to one or several polypeptides encoded by the diabetes-associated alleles of this invention. Status and/or function of a biological network and/or a metabolic pathway can be assessed e.g. by measuring amount or composition of one or several polypeptides or metabolites belonging to the biological network and/or to the metabolic pathway from a biological sample taken from a subject. Risk to develop diabetes is evaluated by comparing observed status and/or function of biological networks and or metabolic pathways of a subject to the status and/or function of biological networks and or metabolic pathways of healthy controls.

Kits (e.g., reagent kits) useful in the methods of diagnosis comprise components useful in any of the methods described herein, including for example, PCR primers, hybridization probes or primers as described herein (e.g., labeled probes or primers), reagents for detection of labeled molecules, restriction enzymes (e.g., for RFLP analysis), allele-specific oligonucleotides, DNA polymerases, RNA polymerases, marker enzymes, antibodies which bind to altered or to non-altered (native) polypeptide encoded by a diabetes-associated allele, means for amplification of nucleic acids comprising one or several diabetes-associated alleles, or means for analyzing the nucleic acid sequence of one or several diabetes-associated allele or for analyzing the amino acid sequence of one or several polypeptides encoded by the diabetes-associated allele, etc. In one embodiment, a kit for diagnosing susceptibility to diabetes can comprise primers for nucleic acid amplification of fragments from a diabetes-associated allele.

### Monitoring Progress of Treatment

The current invention also pertains to methods of monitoring the effectiveness of a treatment of diabetes described herein based on the expression (e.g., relative or absolute expression) of one or more diabetes-associated alleles. The mRNA, or polypeptide it is encoding or biological activity of the encoded polypeptide can be measured in a tissue sample (e.g. peripheral blood sample or adipose tissue biopsy). An assessment of the levels of expression or biological activity of the polypeptide can be made before and during treatment with therapeutic agents known to be useful or with agents examined for their therapeutic activity.

Alternatively the effectiveness of a treatment of diabetes can be followed by assessing the status and/or function of biological networks and/or metabolic pathways related to one or several polypeptides encoded by a diabetes-associated allele of this invention. Status and/or function of a biological network and/or a metabolic pathway can be assessed e.g. by measuring amount or composition of one or several polypeptides, belonging to the biological network and/or to the metabolic pathway, from a biological sample taken from a subject before and during a treatment. Alternatively status and/or function of a biological network and/or a metabolic pathway can be assessed by measuring one or several metabolites belonging to the biological network and/or to the metabolic pathway, from a biological sample before and during a treatment. Effectiveness of a treatment is evaluated by comparing observed changes in status and/or function of biological networks and or metabolic pathways following treatment of affected subject with therapeutic agents to the data available from healthy subjects.

For example, in one embodiment of the invention, an individual who is a member of the target population can be assessed for response to treatment with a disease inhibitor, by examining biological activity of polypeptide encoded by a diabetes-associated allele or absolute and/or relative levels of diabetes-associated allele encoding polypeptide or mRNA in peripheral blood in general or in specific cell fractions or in a combination of cell fractions.

The presence of diabetes-associated alleles and other variations may be used to exclude or fractionate patients in a clinical trial who are likely to have involvement of another pathway which cause the disease in order to enrich patients who have pathways involved that are relevant regarding to the treatment tested and boost the power and sensitivity of the clinical trial. Such variations may be used as a pharmacogenetic test to guide the selection of pharmaceutical agents for individuals.

### Primers, Probes and Nucleic Acid Molecules

A probe or primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence.

In preferred embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

The nucleic acid sequences of the polymorphic alleles described in this invention can also be used to compare with endogenous DNA sequences in patients to identify genetic disorders (e.g., a predisposition for or susceptibility to disease or disorder, as described herein), and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents: For example, these sequences can be used to: (i) map their respective genes on a chromosome; and, thus, locate gene regions associated with genetic disease, particularly diabetes and related disorder; (ii) identify an individual from a minute biological sample (tissue typing. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

### Methods of Diagnosing Predisposition or Susceptibility and Treatment Selection

Information obtained using the assays and kits described herein (alone or in conjunction with information on another genetic defect or environmental factor, which contributes to the disease or condition that is associated with the polymorphic alleles) is useful for determining whether a non-symptomatic subject has or is likely to develop diabetes or a specific type of diabetes. In addition, the information can allow a more customized approach to preventing the onset or progression of diabetes. For example, this information can enable a clinician to more effectively prescribe a therapy that will address the molecular basis of diabetes.

In yet a further aspect, the invention features methods for treating or preventing the development of diabetes that is associated with a polymorphic allele in a subject by administering to the subject an appropriate therapeutic agent. In still another aspect, the invention provides in vitro or in vivo assays for screening test compounds to identify therapeutics for treating or preventing the development of diabetes that is associated with polymorphic alleles.

In yet another embodiment, the invention provides a method for identifying an association between a polymorphic alleles and a trait. In preferred embodiments, the trait is susceptibility to diabetes, disease severity, the staging of diabetes or response to a drug. Such methods have applicability in developing diagnostic tests and therapeutic treatments for diabetes. In other preferred embodiments, the drug is an agonist or antagonist phosphofructokinase, particularly platelet phosphofructokinase. The polymorphic alleles are associated with the development, progression, and treatment response of diabetes. Therefore, for example, detection of the polymorphic alleles, alone or in conjunction with another means in a subject can indicate that the subject has or is predisposed to the development of diabetes.

### Correlations Between Treatment and Polymorphic Alleles

The present invention further relates to a method of predicting the response of an individual having a particular polymorphic allele to a particular pharmaceutical agent. As described hereinabove, diabetes poses a major health problem affecting growing numbers of children and adults, particularly in developed countries. Various medicines for diabetes are available, including insulin replacement, enhancers of insulin sensitivity, alpha-glucosidase inhibitors and more. However, due to disadvantages of the currently used therapeutics for diabetes, there is an ongoing, intensive research for new agents and drugs for the treatment of diabetes. Accordingly, there is an ongoing need for means and method for the optimization of clinical trials in terms of selection of the correct population and accurate monitoring of responsiveness.

Thus, the present invention provides a method utilizing the allelic polymorphisms described herein for predicting and evaluating the response of an individual to a specific therapeutic agent, comprising the steps of (a) determining which polymorphic allele is present in an individual at any one or more of the polymorphic sites shown in Table 1 hereinabove, particularly the GVAR237 alleles and (b) administering a pharmaceutical agent or other therapeutic agent that is anticipated to have the most advantageous therapeutic effect and (c) monitoring the effect of the agent.

In order to deduce a correlation between clinical response to a treatment and a polymorphic allele, it is necessary to obtain data on the clinical responses exhibited by a population of individuals who received the treatment, designated hereinafter “the clinical or affected population”. This clinical data may be obtained by analyzing the results of a clinical trial that has already been run and/or the clinical data may be obtained by designing and carrying out one or more new clinical trials. As used herein, the term “clinical trial” means any research study designed to collect clinical data on responses to a particular treatment, and includes but is not limited to phase I, phase II and phase III clinical trials. Standard methods are used to define the patient population and to enroll subjects.

It is preferred that selection of individuals for the clinical population comprises grading such candidate individuals for the existence of the medical condition of interest and then including or excluding individuals based upon the results of this assessment. This is important in cases where the symptom(s) being presented by the patients can be caused by more than one underlying condition, and where treatments of the underlying conditions are not the same. The therapeutic treatment of interest, or the control treatment (active agent or placebo in controlled trials), is administered to each individual in the trial population and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the polymorphic allele for each individual in the trial population is genotyped, which may be done before or after administering the treatment. After both the clinical and polymorphism data have been obtained, correlations between individual response and polymorphic allele content are created.

Correlations may be produced in several ways. In one method, individuals are grouped by their polymorphic allele, and then the averages and standard deviations of continuous clinical responses exhibited by the members of each polymorphism group are calculated. These results are then analyzed to determine if any observed variation in clinical response between polymorphism groups is statistically significant. Statistical analysis methods which may be used are described in L. D. Fisher and G. van Belle, “Biostatistics: A Methodology for the Health Sciences”, Wiley-Interscience (New York) 1993.

One of many possible optimization algorithms is a genetic algorithm (R. Judson, “Genetic Algorithms and Their Uses in Chemistry” in Reviews in Computational Chemistry, Vol. 10, pp. 1-73, K. B. Lipkowitz and D. B. Boyd, eds. (VCH Publishers, New York, 1997). Simulated annealing (Press et al., “Numerical Recipes in C: The Art of Scientific Computing”, Cambridge University Press (Cambridge) 1992, Ch. 10), neural networks (E. Rich and K. Knight, “Artificial Intelligence”, 2nd Edition McGraw-Hill, New York, 1991, Ch. 18), standard gradient descent methods (Press et al., supra), or other global or local optimization approaches (see discussion in Judson, supra) could also be used.

Correlations may also be analyzed using analysis of variation (ANOVA) techniques to determine how much of the variation in the clinical data is explained by different subsets of the polymorphic sites in the polymorphic allele. ANOVA is used to test hypotheses whether a response variable is caused by or correlated with one or more traits or variables (in this case, polymorphism groups) that can be measured (Fisher and van Belle, supra, Ch. 10). These traits or variables are called the independent variables. To carry out ANOVA, the independent variable(s) are measured and individuals are placed into groups based on their values for these variables. In this case, the independent variable(s) refers to the combination of polymorphisms present at a subset of the polymorphic sites, and thus, each group contains those individuals with a given genotype or haplotype. The variation in response within the groups and also the variation between groups are then measured. If the within-group response variation is large (people in a group have a wide range of responses) and the response variation between groups is small (the average responses for all groups are about the same) then it can be concluded that the independent variables used for the grouping are not causing or correlated with the response variable. For instance, if people are grouped by month of birth (which should have nothing to do with their response to a drug) the ANOVA calculation should show a low level of significance. However, if the response variation is larger between groups than within groups, the F-ratio (=“between groups” divided by “within groups”) is greater than one. Large values of the F-ratio indicate that the independent variable is causing or correlated with the response.

The calculated F-ratio is preferably compared with the Critical F-distribution value at whatever level of significance is of interest. If the F-ratio is greater than the Critical F-distribution value, then one may be confident that the individual's genotype or haplotype for this particular subset of the polymorphic allele is at least partially responsible for, or is at least strongly correlated with the clinical response. From the analyses described above, a mathematical model may be readily constructed by the skilled artisan that predicts clinical response as a function of polymorphic allele content. Preferably, the model is validated in one or more follow-up clinical trials designed to test the model.

The identification of an association between a clinical response and a polymorphic allele may be the basis for designing a diagnostic method to determine those individuals who will or will not respond to the treatment, or alternatively, will respond at a lower level and thus may require more treatment, i.e., a greater dose of a drug. The diagnostic method may take one of several forms: for example, a direct DNA test (i.e., genotyping or haplotyping one or more of the polymorphic alleles), a serological test, or a physical exam measurement. The only requirement is that there be a good correlation between the diagnostic test results and the underlying polymorphic allele that is in turn correlated with the clinical response. In a preferred embodiment, this diagnostic method uses the predictive haplotyping method described above.

### Pharmacogenomics

Knowledge of the particular alleles associated with a susceptibility to developing a particular disease or condition, alone or in conjunction with information on other genetic defects contributing to the particular disease or condition allows a customization of the prevention or treatment in accordance with the individual's genetic profile, the goal of “pharmacogenomics”. Thus, comparison of an individual's polymorphic alleles profile to the population profile for diabetes described in Table 1 permits the selection or design of drugs or other therapeutic regimens that are expected to be safe and efficacious for a particular patient or patient population (i.e., a group of patients having the same genetic alteration).

In addition, the ability to target populations expected to show the highest clinical benefit, based on genetic profile can enable: 1) the repositioning of already marketed drugs; 2) the rescue of drug candidates whose clinical development has been discontinued as a result of safety or efficacy limitations, which are patient subgroup-specific; and 3) an accelerated and less costly development for candidate therapeutics and more optimal drug labeling (e. g., since measuring the effect of various doses of an agent as a function of genotype or haplotype is useful for optimizing effective dose). The treatment of an individual with a particular therapeutic can be monitored by determining protein, mRNA and/or transcriptional level. Depending on the level detected, the therapeutic regimen can then be maintained or adjusted (increased or decreased in dose). In a preferred embodiment, the effectiveness of treating a subject with an agent comprises the steps of: (i) obtaining a pro-administration sample from a subject prior to administration of the agent; (ii) detecting the level of expression or activity of a protein, mRNA or genomic DNA in the pro-administration sample; (iii) obtaining one or more post-administration samples from the subject; (iv) detecting the level of expression or activity of the protein, mRNA or genomic DNA in the post-administration sample; (v) comparing the level of expression or activity of the protein, mRNA or genomic DNA in the pro-administration sample with the corresponding protein, mRNA or genomic DNA in the post-administration sample, respectively; and (vi) altering the administration of the agent to the subject accordingly.

### Theranostics

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest and Herceptin (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

### Surrogate Markers

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, nucleic acid tests and imaging modalities). Surrogate endpoints were used first mainly in the cardiovascular area. For example, antihypertensive drugs have been approved based on their effectiveness in lowering blood pressure. Similarly, in the past, cholesterol-lowering agents have been approved based on their ability to decrease serum cholesterol, not on the direct evidence that they decrease mortality from atherosclerotic heart disease. The measurement of cholesterol levels is now an accepted surrogate marker of atherosclerosis. In addition, currently two commonly used surrogate markers in HIV studies are CD4+ T cell counts and quantitative plasma HIV RNA (viral load). In some embodiments of this invention, the polypeptide/polynucleotide expression pattern may serve as a surrogate marker for diabetes and diabetes related disorders and complications, as will be appreciated by one skilled in the art.

### Examples

### Experiments Plan

1. DNA samples from Coriell DNA repositories were used.
2. Control samples were chosen from NINDS control DNA repository. The DNA set was selected such that:
   (a) The patient is Caucasian American
   (b) Age is 55 years or more
   (c) BMI is 28.1 or higher
   (d) The patient has no diabetic relatives
3. Disease samples were chosen from the ADA (American diabetes association) DNA repository. The DNA set was chosen such that:
   (a) The patients have diabetes
   (b) The patients are Caucasian American
   (c) Onset age is 56 years or less
   (d) The patient has at least one first degree diabetic relative

There were 279 control and 271 disease DNA samples. Each sample was genotyped using a PCR with a forward primer having the nucleic acids sequence AGGAAGGTGCCTCTGTGTGTCC (SEQ ID NO:6) and a reverse primer having the nucleic acid sequence ATCACATTCCGGCACAGTGG (SEQ ID NO:7). The PCR products were then sequenced and the resulting chromatograms were analyzed. Genotyping was done by aligning the sequences to the predicted alleles with manual inspection and curation. FIG. 2 shows an example of a mixed sequence (marked as “mix”) and the separated reference (marked as “ref”) and insertion (marked as “ins”) alleles. The sequences are the reverse complement of the alleles in Table 1 and only the variable region is shown with the insertion shown in bold and Italian.

As a further confirmation a second PCR was done with a forward primer having the nucleic acids sequence GGCCAGAATGTTTGCTCCAG (SEQ ID NO:8) and a reverse primer having the nucleic acid sequence ACCCAGGTGGGCCTTAAATG

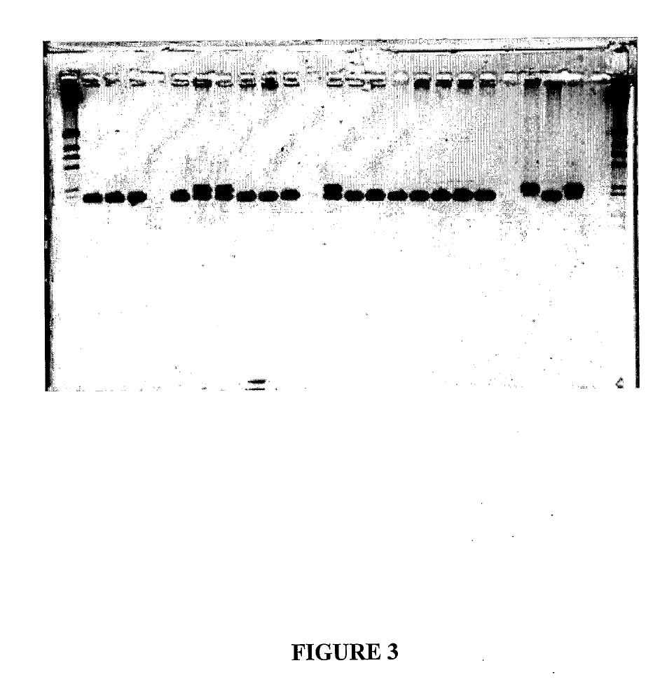

(SEQ ID NO:9). The PCR products of the second reaction, which were about half the size of the products of the first reaction, were then separated using gel electrophoresis. FIG. 3 shows an example of 20 samples with homozygote and heterozygote cases.

Table 2 below summarizes the results for both groups: disease and control (samples obtained from healthy subjects). The column and row named ‘ref’ refer to the reference allele, GVAR237_ref (SEQ ID NO:1) and the column and row named ‘ins’ refer to the insertion allele, GVAR237_ins (SEQ ID NO:2). In the healthy group the following distribution of the alleles was found: 238 homozygotes to the reference allele, 3 homozygotes to the insertion allele and 38 heterozygotes. In the disease group there were 206 homozygotes to the reference allele, 2 homozygotes to the insertion allele, and 63 heterozygotes. The column next to the actual allele numbers presents the percentage of the respective alleles in each group. In the healthy group there were 85.3% homozygotes to the reference allele, while there were only 76.0% homozygotes to the reference allele in the disease group. According to the Fisher exact statistical test the p-value is 0.015 when comparing allele frequencies, and 0.007 when comparing the insertion allele carriers (dominant model). This demonstrates the statistical significance of these results.

TABLE 2

| | |
|---|---|
| ref | ins |

Diabetes Type 2

| | | | | |
|---|---|---|---|---|
| ref | 206 | 76.0% | 63 | 23.2% |
| ins | | | 2 | 0.7% |
| Total | 271 | | | |

Healthy

| | | | | |
|---|---|---|---|---|
| ref | 238 | 85.3% | 38 | 13.6% |
| ins | | | 3 | 1.1% |
| Total | 279 | | | |

P-value allele 0.015

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-31. (canceled)

32. A diagnostic method comprising:
(a) identifying in genetic material of a subject a nucleic acid sequence of a gene encoding platelet phosphofructokinase (PFKP); and
(b) analyzing the nucleic acid sequence for allelic polymorphism indicative of diabetes, predisposition to diabetes, or a condition related thereto.

33. The method according to claim 32, wherein the allele indicative of diabetes or predisposition to diabetes has a correlation with increased risk of diabetes, said allele having a higher appearance frequency in a diabetic population compared to its appearance frequency in a non-diabetic population.

34. The method according to claim 33, wherein the allele correlated with increased risk for diabetes has the nucleic acids sequence as set forth in SEQ ID NO:2.

35. The method according to claim 32, wherein the allele indicative of diabetes has a correlation with reduced risk to diabetes, said allele has the nucleic acid sequence as set forth in SEQ ID NO:1.

36. The method according to claim 32, wherein the presence of the allelic polymorphism is indicative of a subtype of diabetes or predisposition to a subtype of diabetes.

37. The method according to claim 32, wherein the subtype of diabetes is selected from the group consisting of type 1 diabetes, type 2 diabetes, gestational diabetes and maturity onset diabetes of the young (MODY).

38. The method according to claim 32, wherein the condition related to diabetes is one or more conditions selected from the group consisting of obesity; Prader-Willi syndrome; hyperphagia and impaired satiety; anorexia; metabolic disorder; endocrine disorder; gastrointestinal disease; eating disorder; Wolfram syndrome; Alstrom syndrome; mitochondrial myopathy with diabetes; MED-IDDM syndrome; Ipex-linked syndrome; Congenital generalized lipodystrophy (type 2) (Cgl2); Berardinelli-Seip syndrome; and Schmidt syndrome.

39. The method according to claim 32, wherein analyzing the nucleic acid sequence comprises employing at least one oligonucleotide capable of differentiating between the allele having a correlation with increased risk of diabetes having the nucleic acid sequence set forth in SEQ ID NO:2 and the allele having a correlation with reduced risk of diabetes having the nucleic acid sequence set forth in SEQ ID NO:1.

40. The method according to claim 39, wherein analyzing the nucleic acid sequence comprises employing a primer pair comprising a pair of isolated oligonucleotides capable of differentiating between SEQ ID NO:2 and SEQ ID NO:1.

41. The method according to claim 40, wherein the primer pair comprises a forward primer comprises the nucleic acids sequence as set forth in SEQ ID NO:6 and a reverse primer comprises the nucleic acid sequence as set forth in SEQ ID NO:7.

42. The method according to claim 40, wherein the primer pair comprises a forward primer comprises the nucleic acids sequence as set forth in SEQ ID NO:8 and a reverse primer comprises the nucleic acid sequence as set forth in SEQ ID NO:9.

43. An isolated oligonucleotide capable of differentiating between the allele having a correlation with increased risk of diabetes having the nucleic acid sequence set forth in SEQ ID NO:2 and the allele having a correlation with reduced risk of diabetes having the nucleic acid sequence set forth in SEQ ID NO:1.

44. A primer pair capable of differentiating between the allele having a correlation with increased risk of diabetes having the nucleic acid sequence set forth in SEQ ID NO:2 and the allele having a correlation with reduced risk of diabetes having the nucleic acid sequence set forth in SEQ ID NO:1.

45. The primer pair according to claim 44, comprising a pair of from about 10 to about 100 contiguous nucleotides having a nucleic acid sequence selected from the group consisting of SEQ ID NOs:6-7 and SEQ ID NOs:8-9.

46. A method for correlating the effect of a therapeutic agent or a therapeutic regimen useful in the prevention or treatment of diabetes with a diabetes-associated polymorphic allele comprising:
(a) providing a therapeutically effective amount of the agent or employing a therapeutic regimen to a subject having within its genome one diabetes-associated polymorphic allele having a nucleic acid sequence selected from the group consisting of SEQ ID NO:2 and SEQ ID NO:1; and
(b) determining the effect of said therapeutic agent or regimen on at least one phenotypic characteristic of diabetes, wherein a therapeutic agent or regimen altering the at least one phenotypic characteristic is considered useful in preventing or treating diabetes; and
(c) correlating the therapeutic agent or regimen useful in preventing or treating diabetes with the diabetes-associated polymorphic allele of the subject.

47. The method according to claim 46, wherein the agent is administered to a plurality of subjects.

48. The method according to claim 47, further comprising analyzing the effect of the therapeutic agent with regard to allelic combination of the plurality of subjects as to predict the linkage between specific allelic combination and the effect of the therapeutic agent.

49. A method for monitoring the responsiveness of a subject to a candidate therapeutic agent or therapeutic regimen for treating diabetes comprising:
(a) providing a biological sample comprising genetic material from the subject;
(b) determining, in the genetic material, the presence of one diabetes-associated polymorphic allele having a nucleic acid sequence selected from the group consisting of SEQ ID NO:2 and SEQ ID NO:1;
(c) administering to said subject a therapeutically effective amount of the candidate agent or employing the therapeutic regimen; and
(d) determining the effect of said candidate agent or therapeutic regimen on at least one characteristic phenotype of diabetes of said subject;
wherein having a detectable effect indicates said subject having one polymorphic allele selected from SEQ ID NO:2 and SEQ ID NO:1 as responsive to said candidate agent or therapeutic regimen.

50. A method for treating a subject having diabetes or a condition related to diabetes comprising:
(a) providing a biological sample comprising genetic material from the subject;
(b) determining, in the genetic material, the presence of one diabetes-associated polymorphic allele having a nucleic acid sequence selected from the group consisting of SEQ ID NO:2 and SEQ ID NO:1;
(c) selecting a therapeutic agent or a therapeutic regimen useful in the prevention or treatment of diabetes correlated with the diabetes-associated allele of said subject by the method of claim 46; and
(d) administering to said subject the therapeutic agent or regimen selected in step (c).

51. A kit for diagnosing diabetes, predisposition to diabetes or prognosis of diabetes or a related condition comprising reagents, materials and protocols capable of identifying a polymorphic allele of the PFKP gene.

52. The kit according to claim 51, wherein the kit comprises an isolated oligonucleotide capable of differentiating between a polymorphic allele of the PFKP gene having a nucleic acid sequence as set forth in SEQ ID NO:2 and a polymorphic allele having a nucleic acid sequence as set forth in SEQ ID NO:1.

53. The kit according to claim 51, wherein the kit comprises a primer pair capable of differentiating between a polymorphic allele of the PFKP gene having a nucleic acid sequence as set forth in SEQ ID NO:2 and a polymorphic allele having a nucleic acid sequence as set forth in SEQ ID NO:1.

54. The kit according to claim 53, wherein the primer pair comprises a pair of from about 10 to about 100 contiguous nucleotides having a nucleic acid sequence selected from the group consisting of SEQ ID NOs:6-7 and SEQ ID NOs:8-9.
